(19) United States
(12) Patent Application Publication
(10) Pub. No.: US 2006/0167109 A1
(43) Pub. Date: Jul. 27, 2006

(54) Methods of reducing rhinovirus contagion and related compositions

(76) Inventors: Daniel C Pevear, Harleysville, PA (US); Gerald Rhodes, Los Altos, CA (US); Theodore J Nitz, Pottstown, PA (US); Mark A McKinlay, Phoenixville, PA (US); Marc S Collett, Washington, DC (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/504,206

(22) PCT Filed: Feb. 14, 2003
(86) PCT No.: PCT/US03/04598

§ 371 Date: Jun. 23, 2005

Related U.S. Application Data
(60) Provisional application No. 60/357,214, filed on Feb. 14, 2002.
(60) Provisional application No. 60/357,024, filed on Feb. 14, 2002.

(51) Int. Cl. A61K 31/137

(52) U.S. Cl. 514/649

(57) ABSTRACT

The present invention relates to the use of pleconaril to reduce the contagion of rhinovirus virions shed from a host infected with a rhinovirus and thereby reduce disease transmission or re-infection.

### 1. FIELD OF THE INVENTION

The present invention relates to the use of pleconaril to reduce the contagion of rhinovirus virions shed from a host infected with a rhinovirus and thereby reduce disease transmission or re-infection.

### 2. BACKGROUND OF THE INVENTION

Viral respiratory infections (VRI), often referred to collectively as the “common cold,” are the most common cause of acute infectious morbidity. In the United States, more than 85% of the population experiences at least one acute respiratory illness each year. (Benson and Marano, 1998, Vital Health Stat, 10:199). Adults suffer an average of 2 to 3 colds per year; preschool children experience an average of 3 to 7 colds per year. (Turner, 1998, Pediatr Ann, 27:790-795).

Rhinoviruses, a genus of the Picornaviridae family, are the most common human pathogens isolated from patients with viral respiratory infections. Rhinoviruses are implicated in 50% to >80% of upper respiratory tract infections. (Turner, 2001, Antiviral Res, 2001, 49:1-14). In addition to the common cold, rhinoviruses cause a number of other respiratory tract infections and complications. Rhinoviruses have been implicated in acute otitis media, acute sinusitis, acute exacerbations of chronic obstructive pulmonary disease (COPD), and asthma exacerbations in children and adults. (Rotbart and Hayden, 2000, Arch Fam Med, 9:913-922). Specific patient populations such as the elderly, infants, and immunocompromised individuals are particularly susceptible to serious rhinovirus infections that can lead to a significant increase in utilization of medical resources. (Id).

The need for an effective therapy for treatment and a practical means for reducing the transmission of VRI is supported by data from the Center for Disease Control and Prevention (CDC). In 1996 the CDC reported that the common cold was responsible for 56 million days of restricted activity; 27 million office visits; 22 million days of missed work and school, and 45 million bed-ridden days a year in the U.S. (Centers for Disease Control, 1996, Vital Health Stat, 10:200). Annual expenditures in the U.S. for over-the-counter medications for symptomatic relief were approximately $2 billion in the early 1990s, attesting to the intense interest in relief from the morbidity caused by these infections. (See Turner, 1998, supra). In the US, viral respiratory infections are the most frequent reason for inappropriate antibiotic use, which increases the costs associated with VRIs unnecessarily and contributes to the increasing prevalence of antibiotic-resistant bacteria. (See Rotbart and Hayden, supra).

Researchers have faced several challenges in their attempts to develop antiviral agents to treat rhinovirus infections. First, there are more than 100 serotypes of rhinoviruses, which makes vaccine development impractical and has complicated efforts to develop effective antiviral agents with broad activity across all serotypes. (McKinlay, et al., 1992, Annu Rev Microbiol, 46:635-645). Second, for an antirhinoviral compound to be effective, sufficient oral bioavailability and tissue distribution is essential so that an adequate amount of drug reaches and is maintained at the site of infection. Third, as with all acute viral illnesses, treatment must be given early in the course of infection for optimal benefit. Also, because most of the burden of symptoms occurs within the first 3 days of illness, the drug must be capable of reducing the severity of symptoms within the first 24 hours after the initiation of treatment. (See McKinlay, et al., supra; see also Munoz, et al., 2000, Antiviral Res, 46:91-124). Finally, because the clinical manifestation of rhinovirus infection in otherwise healthy individuals is typically an upper respiratory infection that resolves without medical treatment, the drug must have an excellent safety profile to ensure an appropriate benefit-risk ratio. (See McKinlay, et al., supra; see also Diana and Pevear, 1997, Antiviral Chem Chemother, 8:401-408).

Rhinovirus shed in the nasal mucus from infected hosts is a major source of infectious material that results in rhinoviral transmission to uninfected hosts. Rhinovirus infections are spread via pathogen-contaminated respiratory secretions. (Dick, et al., 1987, J Infect Dis, 156:442-448). Hand contact with the nasal mucus is an important mode of human rhinovirus (HRV) transmission in humans. Rhinovirus infections may also be spread by aerosol, direct or indirect contact with virus-contaminated materials (id.). Rhinovirus pathogens can survive on various exogenous surfaces for hours (id.). A method for reducing the infectivity of virions shed from a hosts infected with a rhinovirus therefore would be useful for preventing further spread of VRI to new hosts or the re-infection of a previously infected host.

Progress toward development of a treatment for HRV infections has been substantial in recent years. In vitro studies of several classes of compounds that target viral attachment, uncoating, and viral protease have led to clinical evaluation of the most promising compounds (McKinlay, et al., supra).

Pleconaril, 3-[3,5-dimethyl-4-[3-(3-methyl-5-isoxazolyl) propoxy]phenyl]-5-(trifluoromethyl)-1,2,4-oxadiazole, is the first of a new generation of capsid function inhibitors designed to exhibit good antiviral spectrum of activity, and that has the desired in vivo pharmacokinetics and safety profile. (Rotbart, 2000, Antiviral Chem Chemother, 11:261-271).

U.S. Pat. No. 5,464,848 discloses pleconaril and other related 1,2,4-oxadiazolyl-phenoxyalkylisoxazoles and their use as anti-picornaviral agents.

During human clinical trials, pleconaril has been administered systemically via the oral route for the treatment of viral respiratory infection. See The Antiviral Drugs Advisory Committee Briefing Document—Picovir™ (Pleconaril)—for NDA 21-245 submitted to the U.S. Food & Drug Administration, which is incorporated-by-reference in this application in its entirety. The transfer of significant concentrations of pleconaril from the systemic circulation and from systemic tissues into nasal secretions (nasal mucous) would not be expected in view of the physical and pharmacological properties of pleconaril observed to date in clinical trials.

Pleconaril has a low aqueous solubility (20 ng/ml) and a high log P—that is, it is much more soluble in lipid environments. Thus pleconaril soluble in lipid membranes. Pleconaril is also highly protein bound (>99%) to human plasma proteins.

Nasal mucus is primarily an aqueous environment containing mucopolysaccharides. Mucopolysacharides are glycoproteins, which consist primarily of polysaccharides containing polyglucosamine and polygalactosamine. Partitioning of pleconaril into nasal mucus from systemic tissue would therefore be expected to be extremely low or otherwise be in concentrations those that would impact virus replication.

Citation or identification of any reference in Section 2. of this Application is not an admission that any such reference is available as prior art to the present invention.

### 3. SUMMARY OF THE INVENTION

It has now surprisingly been discovered that pleconaril is effective for reducing rhinovirus contagion by a host infected with one or more rhinoviruses by administering to the infected host an amount of pleconaril or a salt thereof, which is effective to impart to the nasal mucus excreted by the infected host a concentration of pleconaril that is sufficient to reduce infectivity of virions shed in said excreted mucus, and thereby render the infected host less rhinovirally contagious when in contact with an uninfected host.

In a second aspect, the present invention provides a method of protecting an un-infected host from rhinovirus infection due to contagion by a rhinovirus-infected host comprising administering to said infected host an infectivity reducing amount of pleconaril, wherein the infected host is shedding virions of at least one rhinovirus serotype selected from the group consisting of 39, 47, 55, 71, 83, 92, 2, 68, 74, 100, 21, 22, 35, 58, 79, 7, 51, 85, 89, 6, 20, 37, 40, 78, 3, 34, 96, 25, 38, 88, 36, 44, 56, 66, 75, 10, 31, 86, 11, 1B, 14, 33, 46, 62, 63, 49, 57, 80, 90, 28, 19, 24, 61, 73, 9, 82, 17, 30, 43, 18, 76, 67, 29, 53, 70, 81, 23, 64, 32, 65, 50, 12, 1A, 54, 60, 77, 16, 13, 15, 91, 59, 95, 72, 94, 26, 41, 48, 98, 52, 8, 45, 27, and 69, and is exposed to said un-infected host, wherein pleconaril is administered during a time period from about 1 to about 10 hours before such exposure, and preferably from about 2 to about 4 hours before such exposure.

In a third aspect, the present invention provides a method of protecting an un-infected host from rhinovirus infection due to contagion by a rhinovirus-infected host by administering an infectivity reducing amount of pleconaril to the un-infected host, wherein pleconaril is administered during the time period of about 1 to about 10 hours before the prospective contact between the uninfected host and virions of at least one rhinovirus serotype selected from the group consisting of 39, 47, 55, 71, 83, 92, 2, 68, 74, 100, 21, 22, 35, 58, 79, 7, 51, 85, 89, 6, 20, 37, 40, 78, 3, 34, 96, 25, 38, 88, 36, 44, 56, 66, 75, 10, 31, 86, 11, 1B, 14, 33, 46, 62, 63, 49, 57, 80, 90, 28, 19, 24, 61, 73, 9, 82, 17, 30, 43, 18, 76, 67, 29, 53, 70, 81, 23, 64, 32, 65, 50, 12, 1A, 54, 60, 77, 16, 13, 15, 91, 59, 95, 72, 94, 26, 41, 48, 98, 52, 8, 45, 27, and 69.

In a forth aspect, the instant invention provides a method of reducing the infectivity of nasal mucus by treating mucus containing one or more rhinoviruses with an infectivity reducing amount of pleconaril or a salt thereof. In this aspect, the pleconaril may be derived from a source including, but not limited to, oral administration of pleconaril to a host or from tissues impregnated with a pleconaril composition following by collection of excreted nasal mucus from said tissues. Alternatively, the pleconaril may be derived from a source other than nasal administration of a pleconaril formulation or an exogenous combination of nasal mucus and pleconaril.

In a fifth aspect, the present invention provides a composition comprising human nasal mucus and pleconaril.

Other aspects and features of the inventions are described herein below including administering to a rhinovirus infected host an infectivity reducing amount of pleconaril or a salt thereof for the purpose of inhibiting contagion of VRI by such infected host to an un-infected host and reducing the risk of transmitting VRI from an infected host infected with a rhinovirus to an un-infected host not infected with a rhinovirus. Preferably the method is effective at reducing such risk when there is a greater than a 50 percent risk of transmission, greater than a 75 percent risk of transmission, greater than a 95 percent of transmission, and/or greater than a 99 percent risk of transmission.

### 4. DETAILED DESCRIPTION OF THE INVENTION

The present invention provides methods of using pleconaril to reduce the contagion of rhinovirus virions and the corresponding compositions containing pleconaril that are useful for practicing the invention. The methods of the invention summarized above have been found to be effective against rhinovirus serotype selected from the group consisting of 39, 47, 55, 71, 83, 92, 2, 68, 74, 100, 21, 22, 35, 58, 79, 7, 51, 85, 89, 6, 20, 37, 40, 78, 3, 34, 96, 25, 38, 88, 36, 44, 56, 66, 75, 10, 31, 86, 11, 1B, 14, 33, 46, 62, 63, 49, 57, 80, 90, 28, 19, 24, 61, 73, 9, 82, 17, 30, 43, 18, 76, 67, 29, 53, 70, 81, 23, 64, 32, 65, 50, 12, 1A, 54, 60, 77, 16, 13, 15, 91, 59, 95, 72, 94, 26, 41, 48, 98, 52, 8, 45, 27 and 69. These methods may be used to reduce infectivity of rhinovirus progeny virions shed in nasal mucus excreted from the treated host, preferably to the extent that there is no corresponding cultureable rhinovirus in a sample of nasal mucus taken from the host within 1 to 10 hours after administering pleconaril. The nasal mucus concentration of pleconaril obtainable by these methods is at least about the 50 percent minimum inhibitory concentration, preferably at least about the 75 percent minimum inhibitory concentration, more preferably, at least about the 90 percent minimum inhibitory concentration, and most preferably about the 95-99 percent minimum inhibitory concentration. Alternatively, the nasal mucus concentration of pleconaril is more preferably at least about 4.3 μM, and most preferably at least about 12.5 μM. The preferred modes of administering pleconaril in practicing the above-described methods include, without limitation inhalation and topical administration.

The methods also encompass reducing the risk of transmitting VRI from an infected host infected with a rhinovirus to an un-infected host not infected with a rhinovirus. Preferably the method is effective at reducing such risk when there is a greater than a 50 percent risk of transmission, greater than a 75 percent risk of transmission, greater than a 95 percent of transmission, and/or greater than a 99 percent risk of transmission.

Pleconaril exerts its antiviral effect by inhibiting capsid functions that are essential for rhinovirus replication. Specifically, pleconaril integrates within a hydrophobic pocket located within the viral capsid in a manner that blocks virus attachment to cells, uncoating of viral RNA, and infectivity of progeny virions. This pocket is conserved among the majority of rhinoviruses, and explains the broad spectrum of anti-rhinovirus activity exhibited by pleconaril. As described herein, over 90% of the 101 human rhinovirus serotypes were inhibited by pleconaril at drug concentrations that are achievable in the clinic (i.e. in vivo).

The mechanism by which pleconaril acts also explains its selectivity and specificity. Pleconaril shows no activity against unrelated viruses, since these viruses lack the rhinovirus-specific capsid pocket into which pleconaril integrates.

Pleconaril unexpectedly has been found to be excreted in human nasal mucus when pleconaril is administered via an oral formulation. Human patients dosed with an oral pleconaril formulation have been found to excrete nasal mucus that contains pleconaril. Applicants have discovered that oral administration of pleconaril can result in nasal excretion of pleconaril in nasal mucus in concentrations that approach blood plasma concentrations that are considered useful for treating rhinovirus infections. Applicants have also discovered that the unexpected excretion of pleconaril in nasal mucus reduces the infectivity of virions that are co-excreted, or that otherwise come in contact with the excreted pleconaril-nasal mucus compositions. The invention described herein is useful for reducing the infectivity of virions shed by host infected with one or more rhinovirus serotypes.

Absorption and Pharmacokinetic Profile:

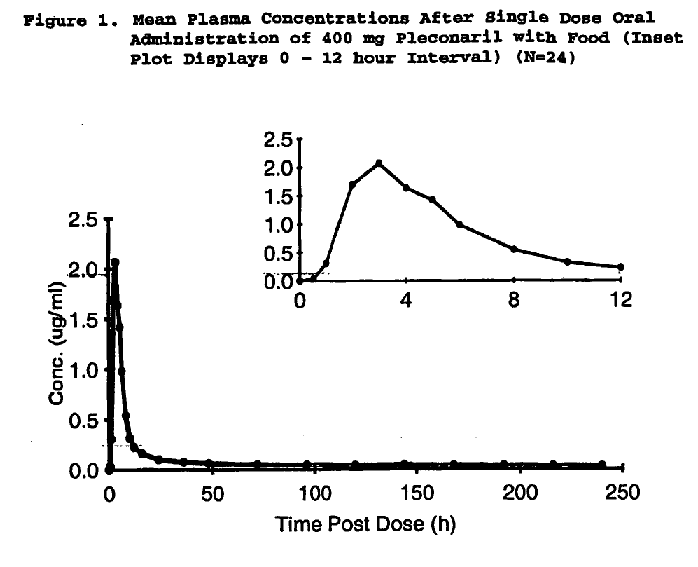

The single dose pharmacokinetic profile of pleconaril is summarized in Table 1. Following oral absorption, pleconaril displays a bi-exponential disposition profile in which a short alpha half-life (2-3 hours) and a long terminal half-life (approximately 180 hours) both contribute significantly to the elimination of pleconaril (FIG. 1).

TABLE 1
Mean Pharmacokinetic Parameters of Pleconaril in Fed
Healthy Young Subjects (N = 24) After Single
Dose Oral Administration of 400 mg Pleconaril.

| Tₘₐₓ | Cₘₐₓ | T_{1/2} | AUC_{0→∞} | CL/F | Vz/F |
|---|---|---|---|---|---|
| (h) | (μg/mL) | (h) | (μg h/mL) | (L/h) | (L) |
| 3.0 | 2.3 | 185.7 | 29.2 | 16.8 | 4113.7 |
| (2.0-5.0) ^{a} | (0.8) ^{b} | (116.6) ^{b} | (14.0) ^{b} | (7.9) ^{b} | (2393.7) ^{b} |

^{a} (Minimum-Maximum)
^{b} (Standard Deviation)

Pleconaril was administered with food in all Phase II and III clinical studies conducted by Applicants. Pleconaril absorption is increased significantly (3-4 fold) when the drug is administered with food.

The pharmacokinetic profile of pleconaril is dose proportional and linear over a plasma concentration range that includes, and exceeds by approximately 2-fold, the plasma concentrations observed in subjects taking the proposed dosing regimen.

Pleconaril has a volume of distribution (Vz/F) that is consistent with significant tissue distribution despite the fact that pleconaril is highly bound (>99%) to plasma protein. Pleconaril is a low systemic clearance drug. Renal clearance contributes insignificantly to the systemic clearance of pleconaril. Less than 1% of the dose is excreted unchanged as pleconaril in urine.

The highly lipophilic nature of pleconaril in combination with the fact that pleconaril is highly bound to plasma protein would lead one of ordinary skill in the art to conclude that little to no pleconaril would partition into an aqueous nasal mucus.

The alpha disposition phase half-life of pleconaril (˜2.8 hours) is most representative of the drug concentration profile in the targeted tissues of interest (e.g., respiratory epithelium) and, thus, is a more relevant pharmacologic half-life for the treatment of VRI. The clinical dosing regimen was designed to maintain antiviral concentrations throughout the dosing interval, considering only the rate of absorption and the alpha disposition half-life. This profile suggested that a three times daily (TID) dosing regimen of pleconaril would be most appropriate for the treatment of VRI. It would also serve to limit the peak to trough plasma concentration range, and thus minimize the total dose administered.

A 400 mg dose of pleconaril maintains antiviral concentrations at or above the concentration that inhibits 90% of the protoype HRV serotypes (90% Minimal Inhibitory Concentration or mic₉₀) for a significant portion of the dosing interval after the first dose of pleconaril. A nasal tissue to plasma concentration ratio of five was estimated for pleconaril from rat tissue distribution studies. Examination of data from individual subjects in early clinical pharmacology studies suggested that subjects with the lowest plasma concentrations at 6 to 8 hours post 400 mg dose would provide therapeutic concentrations in nasal tissue that would meet or exceed the MIC₉₀ of pleconaril (i.e., dosing over inter-patient variability) based on a nasal tissue to plasma partition ratio of five.

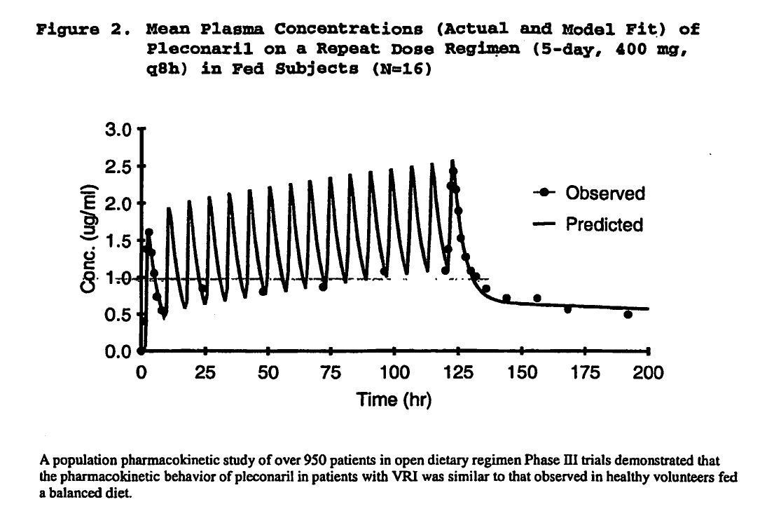

Plasma accumulation of pleconaril was modest (approximately two fold) when pleconaril was administered according to the clinical dosing regimen (400 mg TID for 5 days). The long terminal half-life had only a modest influence on pleconaril plasma concentration-time profiles for the clinical dosing regimen. The repeated dose pharmacokinetic profile of pleconaril was predictable from single dose data and, thus, the pharmacokinetic profile of pleconaril was time independent (FIG. 2).

Pleconaril in Nasal Mucus:

Applicants' discovery that in fact pleconaril can be excreted in measurable amounts in human nasal mucus was unexpected. Further investigation of the discovery revealed that pleconaril nasal mucus concentrations in human clinical trials approach concentrations that would be expected to reduce the infectivity of a significant proportion of virions shed by a host infected by one or more rhinovirus serotypes. Pleconaril-nasal mucus composition described herein may be used to clear infectious material from an infected host while at the same time reducing the risk of re-infection or transmission of infectious viral pathogens to a second host. Applicants have discovered that pleconaril concentrations may be achieved in nasal mucus that reduce the infectivity of rhinovirus virions shed by an infected host. The desirable nasal mucus concentrations of pleconaril may be advantageously obtained by the appropriate oral administration of pleconaril. In the case where desirable mucus concentrations are obtained in an uninfected host, then therein a prophylactic pleconaril nasal mucus composition is created that is useful for inhibiting transmission of infectious rhinovirus virions to the same.

The pleconaril nasal mucus compositions described herein may be advantageously used in healthy individuals as well as unhealthy individuals, or individuals that are infected with a rhinovirus or are not infected with a rhinovirus. The compositions may be used by any individual that desires to inhibit, prevent and/or avoid infection by one or more rhinovirus serotypes, or otherwise desires to reduce the infectivity of rhinovirus virions shed in nasal mucus compositions. The compositions may be used by individuals or health organizations that desire to inhibit, prevent and/or avoid the transmission of rhinovirus infections on a small or global scale, by reducing the infectivity of rhinovirus shed by an infected host.

An infectivity-reducing amount of pleconaril may be achieved by more than one type of oral administration protocol. Single dosing may be desirable in certain situations where short term reduction in infectivity is desired or short term protection against infection is necessary. Multiple dosing regimes may be appropriate for longer term continuous reduction in the infectivity of shed virions.

Antiviral Activity of Pleconaril Against HRV Serotypes:

a) Effect of Pleconaril on Cell Growth

To properly evaluate antiviral activity in cell culture assays, the 50% cytotoxic concentration (CC₅₀) of the test compound was first determined so that any inhibitory effects on virus replication could be distinguished from effects due to compound cytotoxicity. The CC₅₀ of pleconaril was determined using a methyltetrazolium dye-based assay of cell growth (Pevear, et al., 1999, Antimicrob. Agents Chemother., 43, 2109-2115). Since the CC₅₀ consistently fell between 12.5 and 25 μM, the former value was considered to be the highest level of drug testable in these assays.

b) Determination of Antiviral Activity of Pleconaril Against HRV Serotypes

To determine the spectrum of activity and potency of pleconaril against the 101 serotypes of HRVs, each virus was tested in a cell culture assay that measured the drug's efficacy against the cytopathic effects of the viruses, in an infected cell monolayer. As shown in Table 7, the replication of 93 of 101 HRVs was inhibited by pleconaril in the drug concentration range of 0.01 to 6.8 μM. Pleconaril inhibited 50% of the HRV serotypes (50% minimal inhibitory concentration or MIC₅₀) at a drug concentration of ≦0.2 μM (0.08 μg/ml), 80% (MIC₈₀) at a drug concentration of ≦0.78 μM (0.3 μg/ml) and 90% (MIC₉₀) at a drug concentration of ≦4.3 μM (1.6 μg/ml).

c) Effect of Pleconaril on Virus Yield in a Single Cycle of Growth

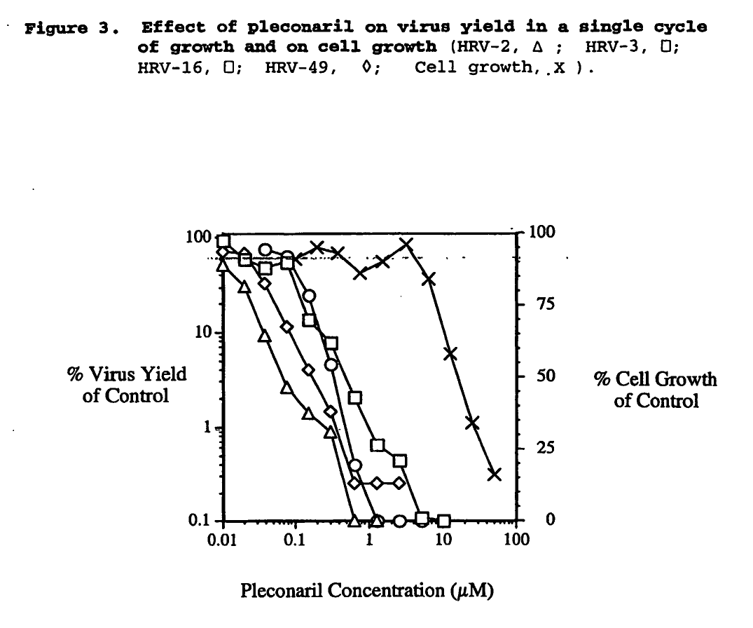

To assess the inhibition by pleconaril of HRV production from a single cycle of virus replication, virus yield experiments were performed. Cells were infected at a multiplicity of infection (MOI) of 1 infectious virion per cell with virus that had been pre-treated with pleconaril at various drug concentrations. After 12 hours of infection (a single replication cycle), the amount of virus produced was quantified. Data for four serotypes is presented in FIG. 3. Pleconaril inhibited virus production in a concentration dependent manner. The yield of progeny virus was reduced by >99% for all four viruses at nontoxic drug concentrations.

d) Antiviral Specificity of Pleconaril

To determine whether pleconaril's antiviral effect in cell culture was specific for HRVs, the drug was tested against 10 other human viral pathogens in viral cytopathic effect assays. All ten viruses were found to be insensitive to pleconaril (IC₅₀ values >12.5 μM; see Table 7).

Mechanism of Action of Pleconaril Against HRV:

a) Inhibition of Rhinovirus Replication

Pleconaril inhibits the replication of 92% (93 of 101) of all prototypic HRV serotypes. Pleconaril inhibits 50% of the HRV serotypes at drug concentrations of ≈0.08 μg/mL (0.2 μM) (50% minimal inhibitory concentration or MIC₅₀) and 90% of the serotypes (MIC₉₀) at 1.6 μg/mL (4.3 μM).

To investigate the mechanism(s) by which pleconaril inhibits HRV replication, six HRVs were selected for further study: HRV-1A, 2, 3, 14, 50 and 89. The sensitivities of these viruses to pleconaril are summarized in Table 2.

TABLE 2
Sensitivities of study viruses to pleconaril
in a viral cytopathic effect assay.

| Rhinovirus | IC₅₀ (μM) ± SD |
|---|---|
| HRV1A | 0.43 ± 0.17 |
| HRV2 | 0.03 ± 0.03 |
| HRV3 | 0.09 ± 0.04 |
| HRV14 | 0.16 ± 0.09 |
| HRV50 | 0.35 ± 0.07 |
| HRV89 | 0.05 ± 0.01 |

Each IC₅₀ value represents the mean of at least two independent determinations with standard deviations (±SD).

b) Time of Drug Addition

To determine the stage in the virus replication cycle that is affected by pleconaril, time of drug addition studies were conducted. Pleconaril was added to cells either during viral attachment or at various times thereafter. Viral replication was allowed to proceed for a single cycle of growth (10-12 hours), at which time the cells were lysed by a freeze-thaw cycle, residual pleconaril was removed by chloroform extraction, and virus yield was determined in the cytopathic effect assay. A 2 μM concentration of pleconaril inhibited greater than 90% of the yield of HRV3 from a single cycle of growth if present during the virus attachment period. Delaying drug addition until after attachment (“0” hour timepoint) resulted in less than a 1 log₁₀ reduction in virus yield. These results indicate that pleconaril exerts its antiviral effect at an early stage of the viral replication cycle.

c) Direct Interaction of Pleconaril with Virions

Since the time of addition experiments indicated that pleconaril acted early in the viral replication cycle, further experiments were conducted to investigate whether pleconaril could interact directly with infectious virions. The experiments were designed in two ways, so as to address two primary questions.

The first question presented is whether pleconaril could bind to human rhinoviruses irreversibly. Various HRVs were incubated overnight at 4° C. with pleconaril at a concentration approximately 10 times the IC₅₀ value. Parallel samples were incubated in medium with 0.5% dimethyl sulfoxide (DMSO) alone. The next day, samples were diluted 10,000-fold into pleconaril-free medium and incubated an additional 24 hrs at 4° C., followed by titration by plaque assay. Table 3 contains the data that show that the infectivity of HRV serotypes 14, 50, and 89 was not fully recovered after drug dilution, even though the drug concentration in the medium was 100-fold below the IC₅₀ value for each respective virus.

TABLE 3
Reversibility of pleconaril binding

| | % Recovery of input |
|---|---|
| HRV serotype | infectious virus (% SD) |
| 1A | 85.4 (52.8) |
| 2 | 65.8 (51.3) |
| 3 | 92.8 (40.4) |
| 5 | 118 (53.5) |
| 14 | 16.4 (33.9) |
| 50 | 11.9 (40) |
| 89 | 9.9 (100) |

The results indicate that pleconaril interacts directly with the virion and binds irreversibly to the indicated HRV serotypes. For these viruses, pleconaril is essentially virucidal. For the remaining HRVs tested (HRV-1A, 2, and 3; Table 3), binding of pleconaril was reversible upon dilution.

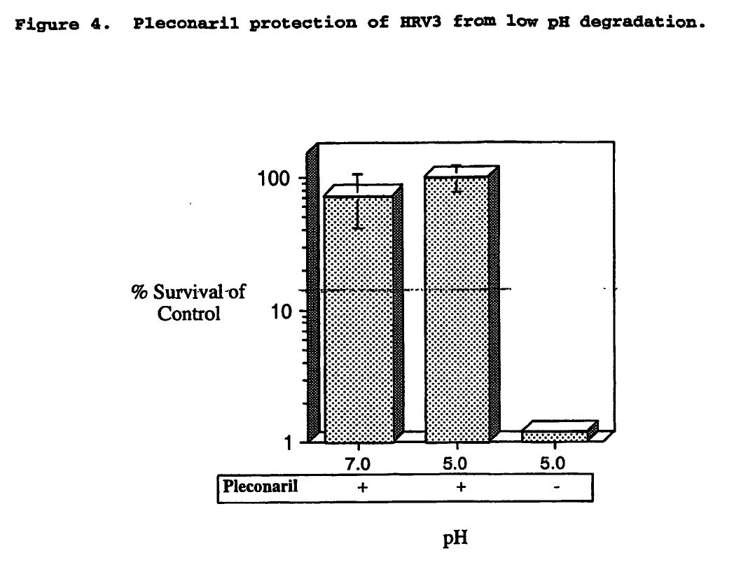

As a second indication of the direct interaction of the drug with the virion capsid, an experiment was conducted to examine the ability of pleconaril to protect HRV3 from low pH inactivation. Pleconaril was found to protect HRV3 from low pH-induced degradation (FIG. 4).

These results indicate that pleconaril interacts directly with the rhinovirus capsid.

d) Inhibition of Attachment

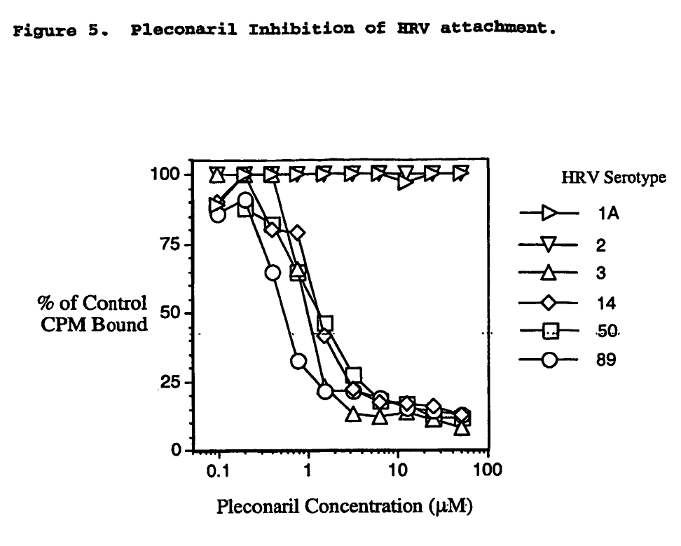

The ability of pleconaril to affect the attachment to cells of several HRV serotypes was examined. ³⁵S-radiolabeled virus was incubated with susceptible cells for 1 hour in the presence of various concentrations of pleconaril. Cell-associated virus was quantified by direct scintillation counting of cell lysates after removal of unbound virus. As shown in FIG. 5, pleconaril inhibited the attachment of several HRV serotypes in a drug

In particular, the attachment to cells of HRVs that use ICAM-1 cellular receptors was inhibited (HRV-3, 14, 50, and 89). (See Staunton, et al., 1989, Cell, 56:849-853; see also Greve, et al., 1989, Cell, 56:839-847). HRVs that use the LDL receptor (HRV-1A and 2) (see Hofer, et al., 1994, Proc Natl Acad Sci USA, 91:1839-1842) were able to bind to HeLa cells normally in the presence of pleconaril. These results indicate that, for certain rhinoviruses, attachment inhibition is one mechanism by which pleconaril inhibits rhinovirus replication. Note, “inhibition of attachment” by pleconaril is not the only operable mechanism for reducing the infectivity of HRV virions, as will appear from discussion below regarding inhibition of uncoating.

e) Inhibition of Uncoating

Pleconaril appeared to inhibit the attachment of only a subset of rhinovirus serotypes. However, the drug exhibits broad-spectrum antirhinovirus activity in cell culture, including the inhibition of virus serotypes not blocked at the attachment stage. These observations suggest that pleconaril may affect rhinoviruses more generally at an early event other than virion attachment.

Following attachment to susceptible cells, rhinoviruses are endocytosed and disassembled or uncoated in order to release the viral RNA for subsequent gene expression and RNA replication. To determine if pleconaril affected the uncoating stage of the virus infection cycle, two types of experiments were carried out. In the first approach, an experiment was conducted to follow directly the fate of intact ³⁵S-radiolabeled (156S) virions attached to cells that were then allowed to proceed through virion disassembly (uncoating) in either the absence or presence of pleconaril. HRV3 virus was chosen for this experiment, as it is representative of the respective genera, and because binding of pleconaril was fully reversible with dilution, minimizing the possibility of drug carryover issues.

Radiolabeled virus was allowed to attach to cells for 1 hour in the absence of pleconaril at room temperature, which permitted virion attachment but did not allow or minimized virion uncoating. After virion attachment, either pleconaril (2 μM) or DMSO control solution was added to the cultures. After an additional 1 hour, the culture was shifted to 33° C., a temperature that permitted virus uncoating. After 2 hours, cell lysates were prepared and the amount of intact 156S virions was assessed by sedimentation of the lysates on continuous sucrose density gradients.

Under these experimental conditions in the absence of pleconaril, nearly 60% of the attached HRV3 was uncoated as evidenced by the disappearance of the intact 156S virion peak relative to the control. However, in the presence of pleconaril, there was minimal reduction in the amount of attached intact 156S virions.

The results of the uncoating experiment indicate that pleconaril acts at the level of virus uncoating by either preventing or slowing the transition from adsorbed, intact virions to disassembled virus competent for subsequent stages of the virus infection cycle.

The second approach to demonstrate the effect of pleconaril on virus uncoating was indirect, in that it monitored the progression of the viral infection cycle beyond the uncoating stage by measuring virus macromolecular synthesis. Immediately after uncoating, viral gene expression (protein translation) and viral RNA replication begin. However, uncoating must occur before either of these two processes can be initiated. In experiments conducted similarly to the uncoating experiment described above, an experiment was conducted to measure viral RNA synthesis in the presence and absence of pleconaril. At the end of the 2 hours temperature shift, actinomycin D was added to the culture medium to inhibit cellular DNA-dependent RNA synthesis. After 2 hours in the presence of actinomycin D, no cellular RNA synthesis was detectable. At that time, ³H-uridine was added to the medium and the incubation was allowed to proceed 2.5 hours (a total of 6.5 hours post-attachment). Total cellular RNA was then recovered, electrophoresed on an agarose-urea gel and visualized by fluorography.

Radiolabeled viral RNA was observed in the HRV3-infected cells in the absence of pleconaril and not in uninfected cells. In pleconaril-treated cells, HRV3 RNA synthesis was completely blocked. The results are consistent with those of the uncoating experiments, and indicate that pleconaril blocks progression of the virus infection cycle beyond the uncoating stage.

f) Inhibition of Multiple Rounds of Viral Replication

The above results showed that pleconaril inhibited early events in the viral replication cycle, including virus attachment to susceptible cells and uncoating of viral RNA, which in turn prevented viral RNA synthesis. The next experiments were designed to answer the question as to whether the addition of pleconaril after the uncoating stage could affect the ability of progeny virions to initiate subsequent rounds of replication, i.e. reduce the infectivity of progeny virions. Cells were infected with HRV3 at a low MOI (0.01 pfu/cell) at room temperature. After removal of unattached virus, fresh medium was added and the cells were shifted to the higher temperature to allow uncoating to proceed. Four hours post-attachment, pleconaril or DMSO alone was added to the cultures, and plates were frozen at 12 hours (upon completion of a single cycle of replication) and 24 hours (upon completion of a second round of replication) post-infection. Virus yield from both rounds of replication was determined in the viral cytopathic effect assay.

When pleconaril was added at a time after uncoating had been completed, it had little effect on the yield of progeny virions from the first round of virus replication. This is consistent with the results from the time of addition experiment. In the absence of pleconaril, a 30- to 50-fold increase in virus yield was observed from the second round of replication relative to the first. However, in the presence of pleconaril, this second round of replication was completely prevented; Virus yields from round 2 decreased relative to round 1 in the presence of 2 μM pleconaril. These results indicate that pleconaril exerts a potent antiviral effect on progeny virions produced in the presence of drug.

### 4.1 Definitions and Abbreviations

The term “contagion” as used herein means transmission of infection by direct contact, droplet spread, or contaminated fomites.

The term “fomites” as used herein denotes objects, such as clothing, towels, and utensils that possibly harbor a disease agent and are capable of transmitting it.

The term “host” as used herein means any organism that is capable of being infected with a virus, or otherwise transmitting a virus particle.

The term “HRV” as used herein means “human rhinovirus.”

The term “HRV particle” denotes a complete rhinovirus particle that is structurally intact that may or may not be infectious, and therefore includes virions as well as other non-infectious viral particles.

The term “infectivity” as used herein means the characteristic of a disease agent that embodies capability of entering, surviving in, and multiplying or causing disease in a susceptible host.

The term “MOI” as used herein denotes “multiplicity of infection” and represents the ratio of infectious virus particles (usually expressed as plaque forming units or “pfu”) per host cell.

The term “pfu” as used herein denotes “plaque forming unit(s).”

The term “Picovir™” is a trademark owned by Sanofi-Synthelabo and exclusively licensed to ViroPharma Incorporated for the marketing of a pleconaril pharmaceutical product in the U.S.A. and Canada.

The term “pleconaril” means the chemical compound pleconaril, 3-[3,5-dimethyl-4-[3-(3-methyl-5-isoxazolyl)propoxy]phenyl]-5-(trifluoromethyl)-1,2,4-oxadiazole, with CAS No. 153168-05-9, also known as VP 63843 and also named 5-[3-[2,6-dimethyl-4-(5-trifluoromethyl-1,2,4-oxadiazol-3-yl)phenoxy]propyl]-3-methylisoxazole in U.S. Pat. No. 5,464,848. Pleconaril has the following chemical formula:

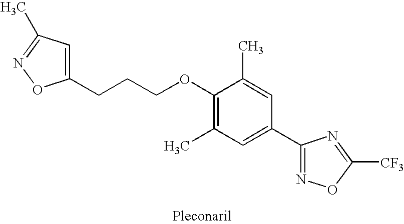

When pleconaril is used to reduce the infectivity of HRV virions via administration to a patient, e.g., to an animal for veterinary use or for improvement of livestock, or to a human for clinical use, pleconaril is preferentially administered in isolated form. As used herein, “isolated” means that pleconaril is separated from other components of either (a) a natural source, such as a plant or cell, preferably bacterial culture, or (b) a synthetic organic chemical reaction mixture. Preferably, pleconaril is purified via conventional techniques. As used herein, “purified” means that when isolated, the isolate contains at least 95%, preferably at least 98%, of a single oxadiazolyl-phenoxyalkylisoxazole compound of the invention by weight of the isolate.

The phrase “pharmaceutically acceptable salt(s),” as used herein includes but is not limited to salts of acidic or basic groups present in the pleconaril compound used in the invention. Pleconaril included in the present compositions includes salts formed with the basic moieties in the pleconaril compound, e.g. salts formed with various inorganic and organic acids found in vivo or otherwise. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. The present compositions of the pleconaril include the situation wherein the acidic moieties of the pleconaril compound form base salts with various pharmacologically acceptable cations or other cations found in vivo or otherwise. Examples of such salts include alkali metal or alkaline earth metal salts and, particularly, calcium, magnesium, sodium, lithium, zinc, potassium, and iron salts.

The term “RT-PCR” as used herein denotes reverse transcription, polymerase chain reaction.

The term “virion” as used herein means the “complete virus particle that is structurally intact and infectious,” also known as the common cold.

The term “VRI” as used herein means “viral respiratory infection.”

### 4.2 Synthesis of Pleconaril

The pleconaril can be obtained via the synthetic methodology described in U.S. Pat. No. 5,464,848.

### 4.3 Therapeutic & Prophylactic Administration and Compositions

Due to the antiviral activity of the pleconaril, it is useful in both veterinary and human medicine. As described above, pleconaril is useful for reducing the infectivity of virions shed from an HRV infected patient by administration thereto.

The invention provides methods of reducing infectivity by administration to a patient of an infectivity reducing effective amount pleconaril, preferably also a therapeutically effective amount for the patient. The patient is an animal, including, but not limited, to a cow, horse, sheep, pig, chicken, turkey, quail, cat, dog, mouse, rat, rabbit and guinea pig and is more preferably a mammal and most preferably a human.

The pleconaril may be administered as such, or in the form of a precursor for which the active agent can be derived, such as a prodrug. A prodrug is a derivative of a compound described herein, the pharmacologic action of which results from the conversion by chemical or metabolic processes in vivo to the active compound.

The pleconaril compositions that may be used to practice the invention are preferably administered orally. Pleconaril can be administered by any other convenient route, for example, by infusion or bolus injection, by absorption through epithelial or mucocutaneous linings (e.g., oral mucosa, rectal and intestinal mucosa) and can be administered together with another biologically active agent. Administration can be systemic or local. Various delivery systems are known, e.g., encapsulation in liposomes, microparticles, microcapsules and capsules and can be used to administer pleconaril, or a pharmaceutically acceptable salt thereof. Methods of administration include but are not limited to intradermal, intramuscular, intraperitoneal, intravenous, subcutaneous, intranasal, epidural, oral, sublingual, intracerebral, intravaginal, transdermal, rectally, by inhalation, or topically, particularly to the ears, nose, eyes, or skin. Modes of administration are left to the discretion of the practitioner and will depend in-part upon the site of the medical condition or disorder. In specific embodiments, it may be desirable to administer pleconaril in conjunction with one or more other anti-viral agents and/or other pharmaceutical compounds (pleconaril combination). This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In certain embodiments, it may be desirable to introduce pleconaril into the central nervous system by any suitable route, including intraventricular, intrathecal and epidural injection. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, pleconaril can be formulated as a suppository, with traditional binders and vehicles such as triglycerides.

In another embodiment, pleconaril can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, pleconaril can be delivered in a controlled-release system. In one embodiment, a pump can be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of pleconaril anti-viral treatment, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) can be used.

The present compositions contain an infectivity-reducing amount, preferably a therapeutically effective amount of pleconaril, or a salt thereof, preferably in purified form, and optionally together with a suitable amount of a pharmaceutically acceptable vehicle so as to provide the form for proper administration to the patient or host.

In a specific embodiment, the term “infectivity-reducing amount” means the amount of drug that will result in the reduction of infectivity of HRV virions. The infectivity-reducing amount that corresponds to a beneficial infectivity-reducing effect by using instant invention may or may not be directly measurable using current analytical techniques for all virus serotypes but nevertheless can be reasonably deduced by scientific extrapolation from other in vitro or in vivo data.

In a specific embodiment, the term “pharmaceutically acceptable” means approved by a regulatory agency of the Federal or a state government, listed in the U.S. Pharmacopeia or other generally recognized pharmacopeia for use in animals and more particularly in humans; or generally regarded by those of skill in the art as being safe to a patient. The term “vehicle” refers to a diluent, adjuvant, excipient, or carrier with which pleconaril or a salt thereof is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, pleconaril compositions are preferably sterile. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” Gennard A. R., (Ed.), Mack Publishing Co., Pennsylvania (1985).

In a preferred embodiment, pleconaril is formulated in accordance with routine procedures as a pharmaceutical composition adapted for intravenous administration to human beings. Typically, pleconaril or salts thereof used in intravenous administration are solutions in sterile isotonic aqueous buffer. Where necessary, the compositions may also include a solubilizing agent. Compositions for intravenous administration may optionally include a local anesthetic such as lidocaine to ease pain at the site of the injection. Generally, the components of the present compositions are supplied either separately or mixed together in unit dosage form, for example, as a dry lyophilized powder or water free concentrate in a hermetically sealed container such as an ampoule or sachette indicating the quantity of active agent. Where pleconaril is to be administered by infusion, it can be dispensed, for example, with an infusion bottle containing sterile pharmaceutical-grade water or saline. Where pleconaril is administered by injection, an ampoule of sterile water for injection or saline can be provided so that the ingredients may be mixed prior to administration.

Compositions for oral delivery may be in the form of tablets, lozenges, aqueous or oily suspensions, granules, powders, emulsions, capsules, syrups, or elixirs, for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract, thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard vehicles such as mannitol, lactose, starch, magnesium stearate, sodium saccharine, cellulose and magnesium carbonate. Such vehicles are preferably of pharmaceutical grade.

The amount of pleconaril, or salts thereof, that will be effective in reducing infectivity of HRV virions will depend on the nature of the infection in the host and can be determined by standard clinical techniques. It is preferable to use a therapeutically effective amount for treating the host when practicing the instant invention. In addition, in vitro or in vivo assays can optionally be employed to help identify optimal dosage ranges. The precise dose to be employed will also depend on the route of administration and the seriousness of the disease or disorder, and should be decided according to the judgment of the practitioner and each patient's circumstances. However, suitable dosage ranges for oral administration are generally about 0.001 milligram to 1000 milligrams of pleconaril (salt equivalents thereof) per kilogram body weight. In specific preferred embodiments of the invention, the oral dose is 0.01 milligram to 20 milligrams per kilogram body weight, more preferably 0.1 milligram to 50 milligrams per kilogram body weight, more preferably 0.5 milligram to 20 milligrams per kilogram body weight, and yet more preferably 1 milligram to 10 milligrams per kilogram body weight. In a preferred embodiment, the oral dose is 5 milligrams of pleconaril per kilogram body weight. The dosage amounts described herein refer to total amounts administered; that is, if more than one compound is administered, the preferred dosages correspond to the total amount of pleconaril administered. Oral compositions preferably contain 10% to 95% active ingredient by weight.

A suitable dosing regime for oral administration is 400 mg of pleconaril three times daily (TID) for 2 to 10 days. On the first day of treatment, three doses should be taken with a minimum of three hours between doses. Pleconaril tablets should be taken with a meal or a snack to improve absorption.

Suitable dosage ranges for intravenous (i.v.) administration are 0.01 milligram to 100 milligrams per kilogram body weight, 0.1 milligram to 35 milligrams per kilogram body weight, and 1 milligram to 10 milligrams per kilogram body weight. Suitable dosage ranges for intranasal administration are generally about 0.01 pg/kg body weight to 1 mg/kg body weight. Suppositories generally contain 0.01 milligram to 50 milligrams of a compound of the invention per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of pleconaril administration are in the range of 0.001 milligram to 1 milligram, depending on the area to which the compound is administered. Effective doses may be extrapolated from dose-response curves derived from in vitro or animal model test systems. Such animal models and systems are well known in the art.

If a compound of the invention were incorporated into a hand-washing procedure or hand-care composition, such procedure or composition may inhibit replication of rhinoviruses and decrease the likelihood of the transmission of the HRV disease.

Where a compound of the invention is administered prior to infection, that is, prophylactically, it is preferred that the administration be performed within about 1 to about 10 hours, preferably, about 2 to about 4 hours prior to exposure to infection of the host animal with the pathogenic virus. Where pleconaril is administered concomitantly to treat a rhinovirus infection, it is preferred that the administration be performed as soon as possible after identification of a need for treatment in the infected patient. A more preferred, embodiment is the administration of pleconaril as soon as the patient or host is infected or suspected of being infected with a HRV.

Other methods will be known to the skilled artisan that may be readily adapted to reducing the infectivity of HRV virions and are therefore also within the scope of the invention.

### 4.4 Combination Therapy

In certain embodiments of the present invention, pleconaril and salts thereof can be used in combination therapy with at least one other therapeutic agent. Pleconaril and the therapeutic agent can act additively or, more preferably, synergistically to reduce the infectivity of one or more rhinoviruses. In a preferred embodiment, pleconaril is administered concurrently with the administration of another therapeutic agent, which can be administered as a component of a composition comprising pleconaril or as a component of a different composition. In another embodiment, a composition comprising pleconaril is administered prior or subsequent to administration of another therapeutic agent. In certain embodiments, where pleconaril is administered in combination with another therapeutic agent that potentially produces adverse side effects including but not limited to toxicity, the therapeutic agent can advantageously be administered at a dose that falls below the threshold at which the adverse side is elicited.

### 5. EXAMPLES AND EXPERIMENTAL PROCEDURES

### 5.1 Measuring Concentration of Pleconaril in Nasal Mucus

a) Stock Solution Preparation

The experiments performed in determining the concentration of pleconaril in nasal mucus involved the preparation of the following stock solutions:

Stock A: 1 mg/ml stock solution was prepared by weighing 12.53 mg of pleconaril and dissolving 12.53 ml of acetonitrile.

Stock B: 0.1 ml of Stock A was added to 0.9 ml of acetonitrile (final concentration 100 μg pleconaril/ml)

Stock C: 0.01 ml of Stock A was added to 0.990 ml of acetonitrile (final concentration 10 μg pleconaril/ml)

Stock D: 0.001 ml of Stock A was added to 1 ml of acetonitrile (final concentration 1 μg pleconaril/ml)

Stock E: 0.1 ml of Stock D was added to 0.9 ml of acetonitrile (final concentration 100 ηg pleconaril/ml)

Stock F: 0.1 ml of Stock E was added to 0.9 ml of acetronitrile (final concentration 10 ηg pleconaril/ml)

b) Standard Preparation

The following volumes of appropriate stock solutions were spiked into 200 μl of pooled placebo nasal mucus to provide pleconaril calibration standards (Table 4)

TABLE 4
Preparation of Pleconaril Nasal Mucus Calibration Standards

| Standard | | |
|---|---|---|
| (ηg pleconaril/ml) | Stock Solution | Volume (μl) |
| 0.1 | F | 2 |
| 1 | E | 2 |
| 10 | D | 5 |
| 25 | D | 5 |
| 50 | D | 10 |
| 75 | D | 15 |
| 100 | D | 20 |

c) Extraction Procedure

Two hundred (200) μl of acetonitrile was added to 200 μl of each nasal mucus standard and sample. The samples and standards were vortex mixed and then centrifuged at 25° C. for 10 minutes at 2500 rpm. 170 μl of supernatant was removed from each standard and sample. Then, 30 μl of water was added. The mixture was vortex mixed and 40 μl was injected for quantitative analysis of pleconaril.

d) Instrument Conditions

Standards and samples were analyzed via LC/MS/MS.
Mass Spec: Micromass Quattro
LC: Alliance 2790
Mobile Phase: 85% Acetonitrile/15% 2 mM Ammonium Acetate (0.2% formic acid)
Flow Rate: 0.5 ml/min
Analytical Column: Luna C18 (2) 5μ, 50×3.00 mm (Phenomenex)
Source: ESI (Electrospray Ionization)
Ion Mode: Positive
MRM transition: 382>82

e) Regression Analysis

The Masslyns quantitation package was used to analyze the results. The method used was a linear (1/× weighted) external standard method of calibration.

f) Results

Table 2 displays the results obtained by linear regression analysis of the nasal mucus pleconaril calibration standards. The correlation coefficient of the regression line was 0.9998. Back-calculated values for the standard curve were within 6% of the nominal value for pleconaril.

TABLE 5
Results of Standard Curve Linear Regression: Back-Calculated
Pleconaril Concentrations and Percent Difference
From Nominal Concentration

| Standard (ng/ml) | Back-Calculated value (ng/ml) | % Difference |
|---|---|---|
| 0.1 | Dropped | NA |
| 1 | 0.94 | 6.0 |
| 10 | 10.49 | 4.9 |
| 25 | 25.50 | 2.0 |
| 50 | 50.53 | 1.1 |
| 75 | 74.49 | 0.7 |
| 100 | 99.06 | 0.9 |

Results from the analysis of pleconaril in fifteen diluted nasal mucus samples from clinical studies 843-043 and 843-044 are shown in Table 6. Studies 843-043 and 843-044 were Phase III clinical trials for the acute treatment of viral respiratory infection (common cold) in which pleconaril was administered for five days (400 mg TID). These samples were selected based on the plasma concentrations measured on Day 3 of 6 (plasma concentrations at or above 1 μg/ml).

Nasal mucus concentrations in the samples analyzed ranged from 1.77 to 79.38 ηg/ml. Nasal mucus samples were diluted with 2 ml of transport media during processing and prior to quantitative analysis. Although the initial volume of the nasal mucus samples was not directly measured, the nasal mucus sample dilution is in the range pf 5-10 fold (nasal mucus samples were generally in the range of 0.2-0.4 ml). Thus, the concentrations in this report significantly underestimate the actual nasal mucus sample concentration of pleconaril prior to dilution.

TABLE 6
Results of Nasal Mucus Sample Analysis from Clinical
Studies 843-043 and 843-044 and related data.

| | | | Pleconaril |
|---|---|---|---|
| | | | Concentration |
| | | Study | (Nasal Mucus) |
| Sub. No. | Day | Number | (ng/ml) |
| 1 | 3 | 43 | 1.20 |
| 1 | 6 | 43 | BQL |
| 2 | 3 | 43 | 26.36 |
| 2 | 6 | 43 | 2.21 |
| 3 | 3 | 43 | 10.22 |
| 3 | 6 | 43 | 33.93 |
| 4 | 3 | 43 | 2.46 |
| 4 | 6 | 43 | 79.38 |
| 5 | 3 | 44 | BQL |
| 5 | 6 | 44 | 1.68 |
| 6 | 3 | 44 | 23.88 |
| 7 | 3 | 44 | 2.51 |
| 7 | 6 | 44 | 1.77 |
| 8 | 3 | 44 | 6.98 |
| 8 | 6 | 44 | 2.43 |

Sub. No. = Patient subject number in clinical trial.
Day = Study Day of clinical trial.
Study Number = Clinical trial study number wherein patients diagnosed with a viral respiratory infection where dosed with 400 mg three times a day for five days.

### 5.2 Measuring Activity of Pleconaril Against HRV Serotypes

The individual pleconaril activities of pleconaril against HRV Serotypes where measured and are listed in Table 7.

TABLE 7
Antiviral activity of pleconaril against HRV serotypes.

| Serotype | IC₅₀ (μM) ± SD |
|---|---|
| 1A | 0.43 ± 0.17 |
| 1B | 0.16 ± 0.03 |
| 2 | 0.03 ± 0.03 |
| 3 | 0.09 ± 0.04 |
| 4 | >12.5 |
| 5 | >12.5 |
| 6 | 0.06 ± 0.02 |
| 7 | 0.05 ± 0.01 |
| 8 | 3.42 ± 2.7 |
| 9 | 0.22 ± 0.08 |
| 10 | 0.13 ± 0.05 |
| 11 | 0.15 ± 0.04 |
| 12 | 0.39 ± 0.12 |
| 13 | 0.58 ± 0.13 |
| 14 | 0.16 ± 0.09 |
| 15 | 0.59 ± 0.29 |
| 16 | 0.57 ± 0.19 |
| 17 | 0.23 ± 0.08 |
| 18 | 0.24 ± 0.1 |
| 19 | 0.20 ± 0.01 |
| 20 | 0.06 ± 0 |
| 21 | 0.04 ± 0.01 |
| 22 | 0.04 ± 0.02 |
| 23 | 0.32 ± 0.01 |
| 24 | 0.21 ± 0.02 |
| 25 | 0.10 ± 0.01 |
| 26 | 1.6 ± 0.6 |
| 27 | 5.9 ± 1.9 |
| 28 | 0.2 ± 0.07 |
| 29 | 0.27 ± 0.07 |
| 30 | 0.23 ± 0.08 |
| 31 | 0.13 ± 0.07 |
| 32 | 0.33 ± 0.13 |
| 33 | 0.16 ± 0.03 |
| 34 | 0.09 ± 0.02 |
| 35 | 0.04 ± 0.01 |
| 36 | 0.11 ± 0.02 |
| 37 | 0.06 ± 0.02 |
| 38 | 0.10 ± 0.04 |
| 39 | 0.01 ± 0 |
| 40 | 0.07 ± 0.03 |
| 41 | 1.75 ± 1.49 |
| 42 | >12.5 |
| 43 | 0.23 ± 0.11 |
| 44 | 0.11 ± 0.07 |
| 45 | 4.3 ± 1.83 |
| 46 | 0.16 ± 0.05 |
| 47 | 0.01 ± 0 |
| 48 | 1.8 ± 0.63 |
| 49 | 0.17 ± 0.06 |
| 50 | 0.35 ± 0.07 |
| 51 | 0.05 ± 0.03 |
| 52 | 2.49 ± 1.21 |
| 53 | 0.28 ± 0.06 |
| 54 | 0.46 ± 0.11 |
| 55 | 0.02 ± 0.02 |
| 56 | 0.11 ± 0.06 |
| 57 | 0.17 ± 0.05 |
| 58 | 0.04 ± 0.03 |
| 59 | 0.78 ± 0.34 |
| 60 | 0.48 ± 0.14 |
| 61 | 0.21 ± 0.08 |
| 62 | 0.16 ± 0.06 |
| 63 | 0.16 ± 0.08 |
| 64 | 0.32 ± 0.24 |
| 65 | 0.33 ± 0.1 |
| 66 | 0.11 ± 0.07 |
| 67 | 0.25 ± 0.11 |
| 68 | 0.03 ± 0.02 |
| 69 | 6.77 ± 3.14 |
| 70 | 0.30 ± 0.19 |
| 71 | 0.02 ± 0.01 |
| 72 | 0.94 ± 0.3 |
| 73 | 0.21 ± 0.09 |
| 74 | 0.03 ± 0 |
| 75 | 0.11 ± 0.06 |
| 76 | 0.24 ± 0.29 |
| 77 | 0.55 ± 0.29 |
| 78 | 0.08 ± 0.04 |
| 79 | 0.04 ± 0.06 |
| 80 | 0.17 ± 0.03 |
| 81 | 0.30 ± 0.07 |
| 82 | 0.22 ± 0.19 |
| 83 | 0.02 ± 0.01 |
| 84 | >12.5 |
| 85 | 0.05 ± 0.02 |
| 86 | 0.13 ± 0.1 |
| 87 | >12.5 |
| 88 | 0.10 ± 0.04 |
| 89 | 0.05 ± 0.01 |
| 90 | 0.19 ± 0.06 |
| 91 | 0.73 ± 0.39 |
| 92 | 0.02 ± 0.01 |
| 93 | >12.5 |
| 94 | 1.1 ± 0.44 |
| 95 | 0.81 ± 0.51 |
| 96 | 0.09 ± 0.05 |
| 97 | >12.5 |
| 98 | 1.8 ± 1.62 |
| 99 | >12.5 |
| 100 | 0.03 ± 0.01 |
| Other Viruses | |
| RSV-A | >12.5 |
| HSV-2 | >12.5 |
| FLUA | >12.5 |
| DEN-2 | >12.5 |
| HAdV-5 | >12.5 |
| Corona | >12.5 |
| Mumps | >12.5 |
| MeV | >12.5 |
| REOV-1 | >12.5 |
| HPIV-3 | >12.5 |

a) Cells and Viruses

HeLa (WIS) cells were obtained from Dr. Roland Rueckert, University of Wisconsin, Madison, Wis. All other cells and viruses were obtained from the American Type Culture Collection (ATCC), Rockville, Md.

b) Drug Solution and Cell Toxicity (MTT) Assay

Pleconaril was solubilized in dimethyl sulfoxide (DMSO). After dilution to the desired drug concentration, the final DMSO concentration in assays was 0.25%. The toxicity of pleconaril in cell culture was determined in an MTT-based assay as previously described (Pevear et al., 1999, supra), except that the incubation conditions were 33° C. and 2.5% CO₂. The 50% cytotoxic concentration (CC₅₀) of pleconaril was defined as the highest concentration of compound that resulted in ≦50% cell growth compared to a no-drug control.

c) Virus Cytopathic Effect Assay for HRVs

HRV stocks were grown in HeLa (WIS) cells in 150 cm² flasks, frozen/thawed 3 times, aliquoted and stored frozen at −80° C. The virus inoculum to be used in the assay for each virus was determined as follows. HeLa (WIS) cells were seeded on 96-well tissue culture plates (Costar, 3598) at a density of 2.8×10⁴ cells/well in M199 medium (Sigma) supplemented with 5% heat-inactivated newborn bovine serum. Twenty-four hours later, serial 0.5 log₁₀ dilutions of each virus stock were plated in octuplicate onto the cells in complete M199 medium (5% heat-inactivated fetal bovine serum (FBS), 0.15 mM MgCl₂, and 7.5 μg/mL diethylamino-dextran (DEAE-dextran)). The plates were incubated for 3 days at 33° C. in a humidified, 2.5% CO₂ atmosphere, then fixed with 5% glutaraldehyde and stained with 0.1% crystal violet. After rinsing and drying, the optical density of the wells was read at a wavelength of 570 nm (OD₅₇₀) on a Bio-Tek model EL-310 plate reader. The highest dilution of virus that resulted in an OD₅₇₀ reading ≦15% of the cell culture control value was used in drug sensitivity testing. Drug sensitivity of HRVs was determined as previously described (Pevear et al., 1999, supra), except that incubation conditions were at 33° C. in complete M199 medium and 2.5% CO₂. The 50% inhibitory concentration (IC₅₀) was defined as the concentration of pleconaril that protected 50% of the cell monolayer from virus-induced cytopathic effect.

d) Virus Yield Reduction Assay

Virus was pretreated with various concentrations of pleconaril for 1 hour at 4° C. Confluent, 1-day-old monolayers of Hela (WIS) cells in 6 well plates were infected with virus at a multiplicity of infection (MOI) of approximately 1 plaque-forming unit per cell. After a 1 hour attachment period at 33° C., the inoculum was removed, the monolayers were washed 3 times with Dulbeccos modified phosphate buffer (DPBS)(JRH Biosciences, Lenexa, Kans.) and overlaid with 3 ml of media containing either the appropriate concentration of pleconaril or drug solvent (0.25% DMSO) alone. Incubation was continued for 12 hours at 33° C. and 2.5% CO₂. All plates were frozen/thawed 3 times at −80° C. prior to quantification of virus yield in the virus cytopathic effect assay.

e) Virus Cytopathic Effect Assays for Other Viruses

The sensitivity of non-picornaviruses to pleconaril was determined in virus cytopathic effect assays similar to the HRV assay for the following viruses: respiratory syncytial virus, strain Long (RSV-A) and herpes simplex virus type II, strain Curtis (HSV-2) on HEp2 cells; influenza virus, strain WSN (FLUA) on MDBK cells; dengue virus type 2, strain New Guinea (DEN-2), and measles virus, strain Edmonston (MeV) on Vero cells; human adenovirus, type 5 (HAdV-5) on HeLa cells (at 33° C.); human coronavirus, strain 229E (Corona) on MRC-5 cells (at 33° C.); and mumps virus, strain Enders (Mumps), reovirus type 1, strain Lang (REOV-1), and parainfluenza virus type 3, strain C243 (HPIV-3) on LLC-MK_{2d} cells. All specificity assays were performed at 37° C. and 5% CO₂ except where noted above.

### 5.3 Mechanism of Action Experiments

a) Cells and Viruses:

All cell lines were obtained from the American Type Culture Collection (Rockville, Md.), except HeLa Ohio (WIS) cells, which were a gift of Roland Rueckert at the University of Wisconsin, Madison. All viruses were purchased from the American Type Culture Collection. Human rhinoviruses were grown on HeLa cells using M199 medium (GibcoBRL, Grand Island, N.Y.) supplemented with 5% fetal bovine serum (FBS, PAA Laboratories, Parker Ford, Pa.) and antibiotics. Human rhinoviruses were grown at 33° C. in a 2.5% CO₂ atmosphere with 95% humidity.

b) Radiolabeling of Viruses:

Rhinoviruses were radiolabeled with ³⁵S-ProMix (Amersham Pharmacia Biotech, Piscataway, N.J.) and purified as previously described (Pevear et al., 1989, J Virol, 63, 2002-2007).

c) Thermal and pH Inactivation:

Approximately 107 plaque forming units (PFU) of virus were incubated with 0.5% DMSO or 2 μM pleconaril (in 0.5% DMSO) overnight at 4° C. For HRV3, samples were diluted into sodium acetate buffer (1 M, pH 5.2 or pH 7.0) for 30 minutes on ice followed by neutralization with 2 volumes of 1M Tris-HCl, pH 8.0. All samples were extracted twice with chloroform prior to titration in the viral cytopathic effect assay on HeLa cells (see below).

d) Reversibility of Binding:

Approximately 106 PFU of virus were incubated with 0.5% DMSO or a pleconaril (in 0.5% DMSO) concentration approximately 10-times that which resulted in 50% inhibition of virus growth in a viral cytopathic effect assay (the 50% inhibitory concentration or IC₅₀) at 4° C. After 24 hours, virus samples were diluted 10,000-fold in culture medium and incubated an additional 24 hours at 4° C. Virus titers in the DMSO- and pleconaril-treated samples were then determined by plaque assay in the appropriate cell line as described below.

e) Time of Drug Addition:

Twenty-four well plates (Costar 3524) of HeLa cells were infected in duplicate with 250 μL of HRV3 in M199 medium with 5% FBS at a multiplicity of infection (MOI) of approximately 1 PFU/cell for 1 hour at room temperature. During a treatment (the “−1 h” timepoint), one set of duplicate wells for each virus received 2 μM pleconaril, while the remaining wells received DMSO (0.5% final concentration). After attachment, the cells were washed twice with 1 mL of DPBS, and then overlaid with fresh medium. Pleconaril (2 μM final concentration) was added to the wells at the indicated times. The plates were incubated at 33° C. (HRV3) for 10 hours, at which time they were frozen at −80° C. Prior to titration in the viral cytopathic effect assay (see below), all samples were extracted twice with chloroform to eliminate any possible drug carryover effects.

f) Attachment Inhibition:

³⁵S-radiolabeled virus was preincubated with various concentrations of pleconaril or 0.5% DMSO alone for 1 hour at room temperature. Cells in 6 well plates were inoculated with 20,000 cpm of ³⁵S-radiolabeled virus/well in 0.5 mL of M199 medium with 5% FBS. Attachment was allowed to proceed for 1 hour at 33° C., at which time the inoculum was removed and the cells washed with 2 mL of medium. The cells were then lysed by a 15 minute incubation on ice in the presence of 250 μL/well of a solution containing detergent 1% Nonident P40 (NP40) and 20 mM chelator, ethylene diamine tetraacetic acid (EDTA) in water. Cell-associated radioactivity was quantified by liquid scintillation spectroscopy in a Wallac Model 1409 Liquid Scintillation Counter (Wallac Oy, Turku, Finland).

g) Uncoating Inhibition:

The protocol of Lee et al., (1993)(Lee W-M., Monroe S. S. and Ruechert, R., 1993, Role of muturation cleavage infectivity of picornaviruses; activation of an infectosome. J. Virol. 67(4):2110-2122) with modifications, was used to monitor uncoating transitions. HeLa cells in 25 cm² flasks (Costar) were infected with 10⁶ CPM of purified, radiolabeled virus for 1 hour at room temperature (HRV3) in 1 mL of DPBS (Cat. No. 59300-78P, JRH Biosciences, Lenexa, Kans.) containing 0.1% bovine serum albumin (PBSA2). Pleconaril (2 μM) or 0.5% DMSO alone was then added, and the incubations were continued for an additional 1 hour at the respective temperatures. The inoculum was then removed and the monolayers were washed twice with 1 mL of PBSA2. The cells were overlaid with 1 mL of PBSA2 with or without pleconaril, and the flasks were temperature shifted to 33° C. (HRV3) for 2 hours. Duplicate flasks were processed without temperature shifting. For processing, the overlay was removed and the cells were lysed by the addition of 750 μL of cell lysis buffer (1% NP40, 0.5% sodium deoxycholate in PBSA2). The lysate was collected and centrifuged at 4000×g in a microcentrifuge for 1 minute to pellet cell debris. The resulting supernatant was layered onto a continuous 5-30% sucrose gradient (w/v) prepared in DPBS with 0.01% BSA and centrifuged at 274,000×g for 70 minutes at 16° C. in an SW41 rotor. The gradients were fractionated from the bottom in approximately 250 μL fraction volumes. Twenty-five μL of each fraction was trichloroacetic acid (TCA)-precipitated onto glass fiber 96 well plates (Millipore Cat. No. MAFBNOB, Millipore Corporation, Bedford, Mass.) and counted by liquid scintillation spectroscopy in a Wallac 1450 MicroBeta 96 well plate reader (Wallac Oy, Turku, Finland).

h) RNA Replication:

HeLa cells in 6 well plates were infected at an MOI of 5 PFU/cell with 1 mL of HRV3 for 1 hour at room temperature in the presence or absence of 2 μM pleconaril. After virus attachment, the incubation was continued for an additional 2 hrs at 33° C. (HRV3). The inoculum was then removed and 3 mL/well of M199 medium with 5% FBS and 5 μg/mL of actinomycin D was added with 2 μM pleconaril or DMSO only (0.5% final concentration). The cells were returned to the incubator for 2 hours, at which time 300 μCi of ³H-uridine (Amersham) was added per well. The incubation was continued for an additional 3 hrs. The supernatant was then removed and the cells were lysed by addition of 1 mL of Trizol reagent (Life Technologies Inc, Grand Island, N.Y.). The RNA was recovered according to the manufacturer's instructions, subjected to electrophoresis on denaturing agarose-urea gels as described (Purchio et al., 1983, J. Virol. 48:320-324) and visualized by fluorography.

i) Multiple Round of Replication:

HeLa cells in 6 well plates were infected with HRV3 at an MOI of 0.001 or 0.01 pfu/cell for 1 hour at room temperature in duplicate. The virus inoculum was then removed and the monolayers washed twice with DPBS. The cells were overlaid with 3 mL of M199 medium with 5% FBS and placed at 33° C. (HRV3). After 4 hrs, half of the wells received pleconaril to a final concentration of 2 μM in 0.5% DMSO, and half received DMSO alone. The cells were returned to the incubator and plates were frozen at 12 and 24 hrs post infection. Virus yield was determined in the cytopathic effect assay after 2 cycles of chloroform extraction.

j) Plaque Assay:

In some experiments, virus titers were determined by plaque assay. Cells in 6 well plates were infected with 1 mL of 10-fold dilutions of virus in duplicate for 1 hour at the appropriate incubation temperature. The inoculum was then removed and the cells were overlaid with 3 mL of agarose overlay medium (Eagles minimum essential medium (Life Technologies, Grand Island, N.Y.) with 50 mM Hepes buffer, pH 7.2, 5% FBS, antibiotics, and 1.5% SeaPlaque (HRVs)). The HRV overlay medium was further supplemented with 30 mM MgCl₂ and 15 μg/mL DEAE dextran. The plates were incubated for 3 days at the appropriate temperature, then fixed with 5% glutaraldehyde and stained with 0.1% crystal violet. After rinsing and drying, plaques were read manually.

k) Titration by Viral Cytopathic Effect Assay:

In some experiments, virus titers were determined in a cytopathic effect assay on the appropriate cell line as previously described (Pevear et al., 1999, supra). Briefly, cells in 96 well plates were infected with serial 0.5 log₁₀ dilutions of virus in quadruplicate in M199 medium with 5% FBS. The plates were incubated at the appropriate temperature for 3 days, then fixed with 5% glutaraldehyde and stained with 0.1% crystal violet. After rinsing and drying, the optical density of the wells was read at a wavelength of 570 nm (OD₅₇₀) on a Vmax Kinetic Microplate Reader (Molecular Devices Corp., Sunnyvale, Calif.). The data were then graphed using a 4-parameter curve fitting program. The virus titer (50% tissue culture infectious dose or TCID₅₀) was defined as the virus dilution resulting in a 50% destruction of the cell monolayer.

1) Antiviral Testing in the Viral Cytopathic Effect Assay:

For sensitivity testing to pleconaril, the appropriate cells were seeded into 96 well plates at a concentration of 4×10⁴/well. After an overnight incubation at 37° C., the medium was removed and the cells were infected with 150 μL of a dilution of virus previously titrated to give ≧85% lysis of the monolayer after 3 days of incubation. The plates were incubated for 1 hour at the appropriate temperature and then overlaid with 50 μL of medium containing serial 2-fold dilutions of pleconaril in 2% DMSO. The final DMSO concentration in the assay was always 0.5%. No virus and no pleconaril controls were included on each plate. All drug concentrations were run in quadruplicate. The plates were then incubated for 3 days at the appropriate temperature followed by processing as described above. The IC₅₀ value is defined as the concentration of pleconaril that protected 50% of the cell monolayer from virus-induced cytopathic effect.

### 5.4 Example 1

The following pharmaceutical formulation may be used—as a prodrug pharmaceutical compositions. A tablet formulation for oral administration containing 200 mg of pleconaril, and the following inactive ingredients: lactose, starch, crospovidone, sodium lauryl sulfate, colloidal silicon dioxide, and magnesium stearate.

### 5.5 Example 2

The pharmaceutical formulation of Example 1 may be administered as follows so as to both reduce the infectivity of HRV virions shed and treat infected patient from VRI.

The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

A number of references have been cited, the entire disclosure of which are incorporated by reference.

What is claimed is:

1. A method of reducing rhinovirus contagion by a host infected with one or more rhinoviruses comprising administering to said host an amount of pleconaril or a salt thereof, which is effective to impart to the nasal mucus excreted by said host a concentration of pleconaril that is sufficient to reduce infectivity of virions shed in said excreted mucus, and thereby render said infected host less rhinovirally contagious when in contact with an uninfected host.

2. A method according to claim 1, wherein the shed virions include at least one rhinovirus serotype selected from the group consisting of 39, 47, 55, 71, 83, 92, 2, 68, 74, 100, 21, 22, 35, 58, 79, 7, 51, 85, 89, 6, 20, 37, 40, 78, 3, 34, 96, 25, 38, 88, 36, 44, 56, 66, 75, 10, 31, 86, 11, 1B, 14, 33, 46, 62, 63, 49, 57, 80, 90, 28, 19, 24, 61, 73, 9, 82, 17, 30, 43, 18, 76, 67, 29, 53, 70, 81, 23, 64, 32, 65, 50, 12, 1A, 54, 60, 77, 16, 13, 15, 91, 59, 95, 72, 94, 26, 41, 48, 98, 52, 8, 45, 27, and 69.

3. A method according to claim 1, wherein the shed virions include at least one rhinovirus serotype selected from the group consisting of 39, 47, 55, 71, 83, 92, 2, 68, 74, and 100.

4. A method according to claim 1, wherein the shed virions include at least one rhinovirus serotype selected from the group consisting of 21, 22, 35, 58, 79, 7, 51, 85, 89, and 6.

5. A method according to claim 1, wherein the shed virions include at least one rhinovirus serotype selected from the group consisting of 20, 37, 40, 78, 3, 34, 96, 25, 38, and 88.

6. A method according to claim 1, wherein the shed virions include at least one rhinovirus serotype selected from the group consisting of 36, 44, 56, 66, 75, 10, 31, 86, 11, and 1B.

7. A method according to claim 1, wherein the shed virions include at least one rhinovirus serotype selected from the group consisting of 14, 33, 46, 62, 63, 49, 57, 80, 90, and 28.

8. A method according to claim 1, wherein the shed virions include at least one rhinovirus serotype selected from the group consisting of 19, 24, 61, 73, 9, 82, 17, 30, 43, and 18.

9. A method according to claim 1, wherein the shed virions include at least one rhinovirus serotype selected from the group consisting of 76, 67, 29, 53, 70, 81, 23, 64, 32, and 65.

10. A method according to claim 1, wherein the shed virions include at least one rhinovirus serotype selected from the group consisting of 50, 12, 1A, 54, 60, 77, 16, 13, 15, and 91.

11. A method according to claim 1, wherein the shed virions include at least one rhinovirus serotype selected from the group consisting of 59, 95, 72, 94, 26, 41, 48, 98, 52, and 8.

12. A method according to claim 1, wherein the shed virions include at least one rhinovirus serotype selected from the group consisting of 45, 27, and 69.

13. A method according to claim 1 wherein said virions are progeny virions.

14. The method according to claim 1 wherein the infectivity of the progeny virions shed in nasal mucus excreted from said host is reduced to the extent that there is no corresponding cultureable rhinovirus in a sample of nasal mucus taken from said host within 1 to 10 hours after administering said infectivity reducing amount of pleconaril.

15. The method according to claim 1, wherein said nasal mucus has a pleconaril nasal mucus concentration of at least about the 50 percent minimum inhibitory concentration.

16. The method according to claim 1, wherein said nasal mucus has a pleconaril nasal mucus concentration of at least about the 75 percent minimum inhibitory concentration.

17. The method according to claim 1, wherein said nasal mucus has a pleconaril nasal mucus concentration of at least about the 90 percent minimum inhibitory concentration.

18. The method according to claim 1, wherein said nasal mucus has a pleconaril nasal mucus concentration of at least about the 95 percent minimum inhibitory concentration.

19. The method according to claim 1, wherein said nasal mucus has a pleconaril nasal mucus concentration of at least about the 99% minimum inhibitory concentration.

20. The method according to claim 1, wherein said nasal mucus has a pleconaril nasal mucus concentration of at least about 0.2 μM.

21. The method according to claim 1, wherein said nasal mucus has a pleconaril nasal mucus concentration of at least about 4.3 μM.

22. The method according to claim 1, wherein said nasal mucus has a pleconaril nasal mucus concentration of at least about 12.5 μM.

23. The method according to claim 1, wherein said pleconaril nasal mucus concentration is less than about 12.5 μM.

24. The method according to claim 1, wherein said pleconaril nasal mucus concentration is less than about 4.3 μM.

25. The method according to claim 1, wherein said pleconaril is administer by inhalation.

26. The method according to claim 1, wherein said pleconaril is administer topically.

27. A method of protecting an un-infected host from rhinovirus infection due to contagion by a rhinovirus-infected host comprising administering to said infected host an infectivity reducing amount of pleconaril, wherein said infected host:
a) is shedding virions of at least one rhinovirus serotype selected from the group consisting of 39, 47, 55, 71, 83, 92, 2, 68, 74, 100, 21, 22, 35, 58, 79, 7, 51, 85, 89, 6, 20, 37, 40, 78, 3, 34, 96, 25, 38, 88, 36, 44, 56, 66, 75, 10, 31, 86, 11, 1B, 14, 33, 46, 62, 63, 49, 57, 80, 90, 28, 19, 24, 61, 73, 9, 82, 17, 30, 43, 18, 76, 67, 29, 53, 70, 81, 23, 64, 32, 65, 50, 12, 1A, 54, 60, 77, 16, 13, 15, 91, 59, 95, 72, 94, 26, 41, 48, 98, 52, 8, 45, 27, and 69, and
b) is exposed to said un-infected host,
wherein pleconaril is administered during a time period from about 1 to about 10 hours before said exposure.

28. The method according to claim 27, wherein the period of administration is from about 2 to about 4 hours.

29. The method according to claim 27, wherein the virions are progeny virions.

30. A method of protecting an un-infected host from rhinovirus infection due to contagion by a rhinovirus-infected host comprising administering an infectivity reducing amount of pleconaril to said un-infected host, wherein pleconaril is administered during the time period of about 1 to about 10 hours before the prospective contact between the uninfected host and virions of at least one rhinovirus serotype selected from the group consisting of 39, 47, 55, 71, 83, 92, 2, 68, 74, 100, 21, 22, 35, 58, 79, 7, 51, 85, 89, 6, 20, 37, 40, 78, 3, 34, 96, 25, 38, 88, 36, 44, 56, 66, 75, 10, 31, 86, 11, 1B, 14, 33, 46, 62, 63, 49, 57, 80, 90, 28, 19, 24, 61, 73, 9, 82, 17, 30, 43, 18, 76, 67, 29, 53, 70, 81, 23, 64, 32, 65, 50, 12, 1A, 54, 60, 77, 16, 13, 15, 91, 59, 95, 72, 94, 26, 41, 48, 98, 52, 8, 45, 27, and 69.

31. A method of inhibiting contagion of VRI by an infected host to an un-infected host comprising reducing the infectivity of progeny rhinovirus virions that are shed from an infected host by administering to said infected host an infectivity reducing amount of pleconaril or a salt thereof.

32. A method of reducing the risk of transmitting VRI from an infected host infected with a rhinovirus to an un-infected host not infected with a rhinovirus, comprising administering to said infected host an infectivity reducing amount of pleconaril or a salt thereof.

33. A method according to claim 32, wherein there is a 50 percent or greater risk of said infected host transmitting a VRI to said un-infected host before administration of pleconaril.

34. A method according to claim 33, wherein said risk is greater than 75 percent.

35. A method according to claim 33, wherein said risk is greater than 95 percent.

36. A method according to claim 33, wherein said risk is greater than 99 percent.

37. A method of inhibiting contagion of VRI in an uninfected host, comprising administering an infectivity reducing amount of pleconaril or a salt thereof to an infected patient infected with one or more rhinoviruses before said uninfected host is exposed to said infected patient.

38. A method according to claim 37, wherein said infectivity reducing amount of pleconaril is administered during a time period from about 1 to about 10 hours before said uninfected host is exposed to said infected patient.

39. A method according to claim 38, wherein the period of administration is from about 2 to about 4 hours.

40. A method of reducing the infectivity of nasal mucus comprising treating mucus containing one or more rhinoviruses with an infectivity reducing amount of pleconaril or a salt thereof.

41. A method of reducing nasal mucus infectivity according to claim 40, wherein said pleconaril is derived from oral administration of pleconaril to a host.

42. A method of reducing nasal mucus infectivity according to claim 40, wherein said pleconaril is not derived from:
a) nasal administration of a pleconaril formulation, or
b) exogenous combination of nasal mucus and pleconaril.

43. A method of reducing according to claim 40, wherein said pleconaril is derived from tissues impregnated with a pleconaril composition and said tissues are used to collect excreted nasal mucus.

44. A method according to claim 37, wherein said pleconaril is administered orally.

45. A method according to claim 44, wherein said infectivity reducing amount of pleconaril is 400 mg three times daily for 5 days.

46. A method according to claim 45, wherein said pleconaril is administered in capsule form.

47. A method according to claim 45 wherein said pleconaril is administered as a tablet.

48. A method according to claim 47, wherein said tablet comprises 200 mg of pleconaril, and the following inactive ingredients: lactose, starch, crospovidone, sodium lauryl sulfate, colloidal silicon dioxide, and magnesium stearate.

49. A composition comprising human nasal mucus and pleconaril.

50. A composition according to claim 49, wherein said pleconaril is derived as a component from nasal epithial cell excretion.

51. The composition according to claim 49, additionally comprising one or more metabolites of pleconaril.

52. The composition according to claim 49, additionally comprising one or more rhinoviruses.

53. The composition according to claim 49, wherein said rhinoviruses is at least one selected from the group consisting of serotype 39, 47, 55, 71, 83, 92, 2, 68, 74, 100, 21, 22, 35, 58, 79, 7, 51, 85, 89, 6, 20, 37, 40, 78, 3, 34, 96, 25, 38, 88, 36, 44, 56, 66, 75, 10, 31, 86, 11, 1B, 14, 33, 46, 62, 63, 49, 57, 80, 90, 28, 19, 24, 61, 73, 9, 82, 17, 30, 43, 18, 76, 67, 29, 53, 70, 81, 23, 64, 32, 65, 50, 12, 1A, 54, 60, 77, 16, 13, 15, 91, 59, 95, 72, 94, 26, 41, 48, 98, 52, 8, 45, 27, and 69.

54. The composition according to claim 49, wherein said rhinoviruses is at least one selected from the group consisting of serotype 39, 47, 55, 71, 83, 92, 2, 68, 74, and 100.

55. The composition according to claim 49, wherein said rhinoviruses is at least one selected from the group consisting of serotype 21, 22, 35, 58, 79, 7, 51, 85, 89, and 6.

56. The composition according to claim 49, wherein said rhinoviruses is at least one selected from the group consisting of serotype 20, 37, 40, 78, 3, 34, 96, 25, 38, and 88.

57. The composition according to claim 49, wherein said rhinoviruses is at least one selected from the group consisting of serotype 36, 44, 56, 66, 75, 10, 31, 86, 11, and 1B.

58. The composition according to claim 49, wherein said rhinoviruses is at least one selected from the group consisting of serotype 14, 33, 46, 62, 63, 49, 57, 80, 90, and 28.

59. The composition according to claim 49, wherein said rhinoviruses is at least one selected from the group consisting of serotype 19, 24, 61, 73, 9, 82, 17, 30, 43, and 18.

60. The composition according to claim 49, wherein said rhinoviruses is at least one selected from the group consisting of serotype 76, 67, 29, 53, 70, 81, 23, 64, 32, and 65.

61. The composition according to claim 49, wherein said rhinoviruses is at least one selected from the group consisting of serotype 50, 12, 1A, 54, 60, 77, 16, 13, 15, and 91.

62. The composition according to claim 49, wherein said rhinoviruses is at least one selected from the group consisting of serotype 59, 95, 72, 94, 26, 41, 48, 98, 52, and 8.

63. The composition according to claim 49, wherein said rhinoviruses is at least one selected from the group consisting of serotype 45, 27, and 69.

64. A composition comprising human nasal mucus and pleconaril, wherein said pleconaril is not derived from exogenous nasal administration.
